(19) United States
(12) Patent Application Publication
(10) Pub. No.: US 2026/0207900 A1
(43) Pub. Date: Jul. 23, 2026

(54) SPINAL TREATMENT APPARATUS AND METHOD

(71) Applicant: HADASIT MEDICAL RESEARCH SERVICES AND DEVELOPMENT LTD., Jerusalem (IL)

(72) Inventors: Andres Arnaldo Vargas, Jerusalem (IL); Uri Hadelsberg, Tel Aviv (IL)

(21) Appl. No.: 19/142,814

(22) PCT Filed: Dec. 26, 2023
(86) PCT No.: PCT/IB2023/063248

§ 371 Date: Jun. 24, 2025

Related U.S. Application Data
(60) Provisional application No. 63/435,480, filed on Dec. 27, 2022.

(51) Int. Cl. A61M 25/10; A61B 17/34; A61M 25/01

(52) U.S. Cl. CPC A61M 25/10; A61B 17/3401; A61M 25/0127; A61M 2025/1079

(57) ABSTRACT

Apparatus is provided including an intradural catheter (22) configured to be passed into a spinal canal of a subject, and to be advanced toward a compressed nerve. An inflatable element (40) disposed in the intradural catheter and configured to be inflated from a collapsed state to an inflated state, such that when in the inflated state the inflatable element (40) is shaped to define an outer wall (42), an inner wall (44) and a cavity (46) at least partially surrounded by the inner wall. The inflatable element is inflated, through the intradural catheter, around the nerve such that (a) the nerve is disposed in the cavity surrounded by the inner wall of the inflatable element, and (b) the outer wall of the inflatable element is inflated outwardly to apply pressure in a direction that is away from the nerve. Other applications are also described.

### CROSS-REFERENCES TO RELATED APPLICATIONS

The present application claims priority from U.S. Provisional Ser. No. 63/435,480 to Vargas et al., filed Dec. 27, 2022, entitled “SPINAL TREATMENT APPARATUS AND METHOD”, which is incorporated herein by reference.

### FIELD OF EMBODIMENTS OF THE INVENTION

Some applications of the present invention generally relate to devices and methods for diagnosis and treatment of spinal and/or cerebral pathologies. More specifically, some applications of the present invention generally relate to minimally invasive devices and methods for diagnosis and treatment of spinal and/or cerebral pathologies.

### BACKGROUND

Spinal pathologies, (including trauma, oncological, infectious, neoplastic and degenerative disease of the spine) encompass a wide range of different disease processes. Similarly, cranial pathologies such as cerebrospinal fluid (CSF) abnormalities, and cranial vascular and oncological disorders, include many disease processes. These diseases may lead to impaired quality of life and to an accelerated aging process. In some cases, these pathologies may be life threatening. MRI is a currently available imaging technique for providing information and delineation of soft-tissue and osseous structures of the spine. However, there are cases in which it is not possible to perform an MRI scan (e.g., in cases of claustrophobia of a subject, hemodynamic instability, and/or a subject with a heart pacemaker) Additionally, MRI interpretation may be highly variable resulting in inaccurate diagnoses and increased false positive, or false negative, rates. There is an ongoing need to provide alternative systems that provide accurate diagnosis and effective treatment of spinal pathologies.

### SUMMARY

There is provided in accordance with some applications of the present invention, apparatus and methods for minimally invasive diagnosis and treatment of spinal pathologies. Typically, the apparatus and methods provided herein are configured for performing diagnosis and treatment of the entire spine, at any location from the lumbar spine to the cranio-cervical region and all the way to the intracranial space, in a minimally invasive manner.

For some applications the apparatus comprises a steerable catheter, e.g., a steerable microcatheter, configured to be navigated to, and access, various locations along a spine of a subject. For example, the catheter comprises an intradural catheter configured to be inserted into an intradural space of the subject and to be further steered within the intradural space to a desired location for the purpose of diagnosis and/or performing a medical procedure at the desired location, while providing real time visualization. Typically, the apparatus further comprises medical devices and/or surgical tools configured for performing medical procedures, such as sampling of tissue, resection, and/or treatment of a conditions such as compressed nerves. These medical devices and/or surgical tools are delivered through the catheter to the desired site (any spinal site and all the way to the intracranial compartment) and are manipulated through the catheter by a treating physician from outside the body of the subject, while providing real time visualization to the treating physician.

For some applications, the apparatus comprises the intradural catheter and an inflatable element configured for treatment of a compressed nerve, e.g., a compressed neural foramen nerve. For some such applications, the intradural catheter is passed into a spinal canal of the subject and advanced toward the compressed nerve in a vicinity of the neural foramen. The inflatable element, e.g., a balloon, is delivered through the catheter in a collapsed state and advanced out of a distal end of the catheter. In an inflated state, the inflatable element is shaped to define an outer wall, an inner wall and a cavity at least partially surrounded by the inner wall. The inflatable element is brought into proximity to the compressed nerve and inflated around the nerve such that the nerve is disposed in the cavity surrounded by the inner wall of the inflatable element, and the outer wall of the inflatable element is inflated outwardly to apply pressure in a direction that is away from the nerve, thereby decompressing the nerve. Typically, the inner wall of the inflatable element does not contact the nerve and does not apply pressure to the nerve.

In accordance with additional applications of the present invention, an inflatable element comprising an electrode is delivered through the catheter and advanced out of a distal end of the catheter in proximity to a compressed nerve in the vicinity of neural foramen. In the inflated state the inflatable element is typically shaped to define a near side and a far side. The inflatable element is typically inflated through the catheter, in the neural foramen in proximity to the nerve, such that the near side of the inflatable element faces the nerve, and the far side of the inflatable element faces away from the nerve. The far side is typically inflated outwardly to apply pressure in a direction that is away from the nerve, thereby decompressing the nerve. The near side typically does not apply pressure to the nerve. The electrode that is coupled to the inflatable element is configured to sense nerve activity of the nerve and is typically coupled to the near side of the inflatable element such that the electrode faces the nerve.

There is therefore provided in accordance with some applications of the present invention, apparatus including:
an intradural catheter configured to be passed into a spinal canal of the subject and to be advanced toward a compressed nerve in the spinal canal; and
an inflatable element disposed at a distal end of the intradural catheter and configured to be inflated from a collapsed state to an inflated state, such that when in the inflated state the inflatable element is shaped to define an outer wall, an inner wall and a cavity at least partially surrounded by the inner wall;
the inflatable element is configured to be inflated, through the intradural catheter, around the nerve such that (a) the nerve is disposed in the cavity surrounded by the inner wall of the inflatable element, and (b) the outer wall of the inflatable element is inflated outwardly to apply pressure in a direction that is away from the nerve.

For some applications, the inflatable element includes a toroidal inflatable element.

For some applications, the inflatable element includes an inflatable balloon.

For some applications, the inner wall of the inflatable element is shaped to define an inner diameter of the inflatable element that is 0.3-0.4 cm.

For some applications, the outer wall is shaped to define an outer diameter of the inflatable element that is 0.6-0.8 cm.

For some applications, the apparatus further includes an imaging device disposed at the distal end of the intradural catheter and configured to provide visualization of an area of the spine that is treated by the apparatus.

For some applications, the intradural catheter includes a flexible catheter and the distal end of the intradural catheter includes a bendable tip configured to be rotated up to 360 degrees.

For some applications, the intradural catheter is configured to allow passage therethrough of a surgical tool selected from the group consisting of: forceps, an electrosurgery tool, a cutting tool, and a suction device.

For some applications, the apparatus further includes a puncturing element configured to puncture spinal dura of the subject to allow the intradural catheter to be passed into the spinal canal, the catheter being configured to be passable through the puncturing element.

For some applications, the intradural catheter is configured to be passed into the spinal canal of the subject and to be advanced toward the compressed nerve in a vicinity of a neural foramen in the spinal canal.

For some applications, the intradural catheter is configured to be passed into the spinal canal of the subject and to be advanced toward the compressed nerve in a vicinity of a spinal cord.

For some applications, the distal end of the intradural catheter includes a magnetic distal tip configured to be imaged by MR and/or CT imaging to detect a location of the distal tip.

There is further provided in accordance with some applications of the present invention, a method for decompressing of a nerve of a subject, the method including:
inserting into a spinal canal of a subject, an intradural catheter that includes an inflatable element disposed at a distal portion of the intradural catheter;
advancing the intradural catheter toward a compressed nerve in a vicinity of a neural foramen in the spinal canal;
inflating, through the intradural catheter, the inflatable element around the nerve such that: (a) the nerve is disposed in a cavity that is at least partially surrounded by an inner wall of the inflatable element, and (b) an outer wall of the inflatable element is inflated outwardly to apply pressure in a direction that is away from the nerve, thereby decompressing the nerve.

For some applications, inflating the inflatable element includes inflating the inflatable element to define a toroidal shape.

For some applications, inflating the inflatable element around the nerve includes inflating the inflatable element such that pressure applied by the outer wall is greater than pressure applied by the inner wall.

For some applications, inflating the inflatable element around the nerve includes inflating the inflatable element such that the inner wall does not apply pressure to the nerve.

For some applications, inflating the inflatable element around the nerve includes inflating the inflatable element such that the inner wall is placed at a distance of 3 mm from the nerve.

For some applications, the method further includes using a puncturing element puncturing spinal dura of the subject to allow passing of the intradural catheter into the spinal canal.

For some applications, the method further includes using an imaging device disposed in the intradural catheter.

For some applications, the method further includes passing a surgical tool out of the distal end of the intradural catheter.

There is still further provided in accordance with some applications of the present invention, a method including:
inserting into a spinal canal of a subject, an intradural catheter that includes an inflatable element disposed at a distal portion of the intradural catheter;
advancing the intradural catheter toward a spinal cord;
inflating, through the intradural catheter, the inflatable element around the spinal cord such that: (a) the spinal cord is disposed in a cavity that is at least partially surrounded by an inner wall of the inflatable element, and (b) an outer wall of the inflatable element is inflated outwardly to apply pressure in a direction that is away from the spinal cord.

For some applications, inflating the inflatable element around the spinal cord includes inflating the inflatable element such that a diameter of the spinal canal is expanded.

For some applications, inflating the inflatable element around the spinal cord includes inflating the inflatable element such that pressure applied by the outer wall is greater than pressure applied by the inner wall.

For some applications, inflating the inflatable element around the spinal cord includes inflating the inflatable element such that the inner wall does not apply pressure to the spinal cord.

For some applications, inflating the inflatable element around the spinal cord includes inflating the inflatable element such that the inner wall is placed at a distance of 3 mm from the spinal cord.

For some applications, the method includes using an imaging device disposed in the intradural catheter.

There is yet further provided in accordance with some applications of the present invention, a method for decompressing of a nerve of a subject, the method including:
advancing an inflatable element toward the nerve of the subject;
inflating the inflatable element around the nerve such that: (a) the nerve is disposed in a cavity that is surrounded by an inner wall of the inflatable element, and (b) an outer wall of the inflatable element is inflated outwardly to apply pressure in a direction that is away from the nerve, thereby decompressing the nerve.

There is still further provided in accordance with some applications of the present invention, apparatus including:
an intradural catheter configured to be passed into a spinal canal of the subject and to be advanced toward a nerve in a vicinity of a neural foramen in the spinal canal;
an inflatable element disposed at a distal end of the intradural catheter and configured to be inflated from a collapsed sate to an inflated state in the neural foramen, such that in the inflated state the inflatable element is shaped to define a near side and a far side; and
at least one electrode coupled to the inflatable element and configured to sense nerve activity of the nerve;
the inflatable element is configured to be inflated, in proximity to the nerve such that (a) the near side faces the nerve and (b) the far side faces away from the nerve, the far side being configured to be inflated outwardly to apply pressure in a direction that is away from the nerve.

For some applications, the electrode is coupled to the near side of the inflatable element.

For some applications, when in the inflated state the near side of the inflatable element does not apply pressure to the nerve.

There is still further provided in accordance with some applications of the present invention, a method including:
passing into a spinal canal of a subject, an intradural catheter that includes an inflatable element disposed at a distal portion of the intradural catheter, and at least one electrode coupled to the inflatable element;
advancing the intradural catheter toward a compressed nerve in a vicinity of a neural foramen in the spinal canal;
using the at least one electrode, sensing activity of the nerve; and
inflating, through the intradural catheter, the inflatable element in the neural foramen such that: (a) a near side of the inflatable element faces the nerve, and (b) a far side of the inflatable element faces away from the nerve and is inflated outwardly to apply pressure in a direction that is away from the nerve.

The present invention will be more fully understood from the following detailed description of embodiments thereof, taken together with the drawings, in which:

### BRIEF DESCRIPTION OF THE DRAWINGS

FIG. 1 is a schematic illustration of an overview of a system comprising an apparatus for diagnosis and treatment of spinal pathologies, in accordance with some applications of the present invention;

FIGS. 2A, 2B, 2C, and 2D are schematic illustrations of an intradural catheter for use in accordance with some applications of the present invention;

FIG. 3A is a schematic illustration of the inflatable element for use with the intradural catheter during medical procedures illustrated in FIG. 3B and FIG. 3C, in accordance with some applications of the present invention;

FIG. 3B is a schematic illustration of a medical procedure being performed using the apparatus comprising the intradural catheter and the inflatable element of FIG. 3A, in accordance with some applications of the present invention;

FIG. 3C is a schematic illustration of a medical procedure being performed using the apparatus comprising the intradural catheter and the inflatable element of FIG. 3A, in accordance with some applications of the present invention;

FIG. 4 is a schematic illustration of a medical procedure being performed using the apparatus comprising the intradural catheter and an inflatable element, in accordance with some applications of the present invention;

FIG. 5 is a schematic illustration of another medical procedure being performed using the apparatus comprising the intradural catheter and an inflatable element, in accordance with some applications of the present invention;

FIG. 6 is a schematic illustration of another medical procedures being performed using the apparatus comprising the intradural catheter and a surgical tool, in accordance with some applications of the present invention;

FIGS. 7A, 7B, and 7C are schematic illustrations of the apparatus comprising the intradural catheter and a medical divider, in accordance with some applications of the present invention; and

FIGS. 7D, 7E and 7F are schematic illustrations of a medical procedure being performed using the apparatus comprising the intradural catheter and the medical divider, in accordance with some applications of the present invention.

### DETAILED DESCRIPTION OF EMBODIMENTS

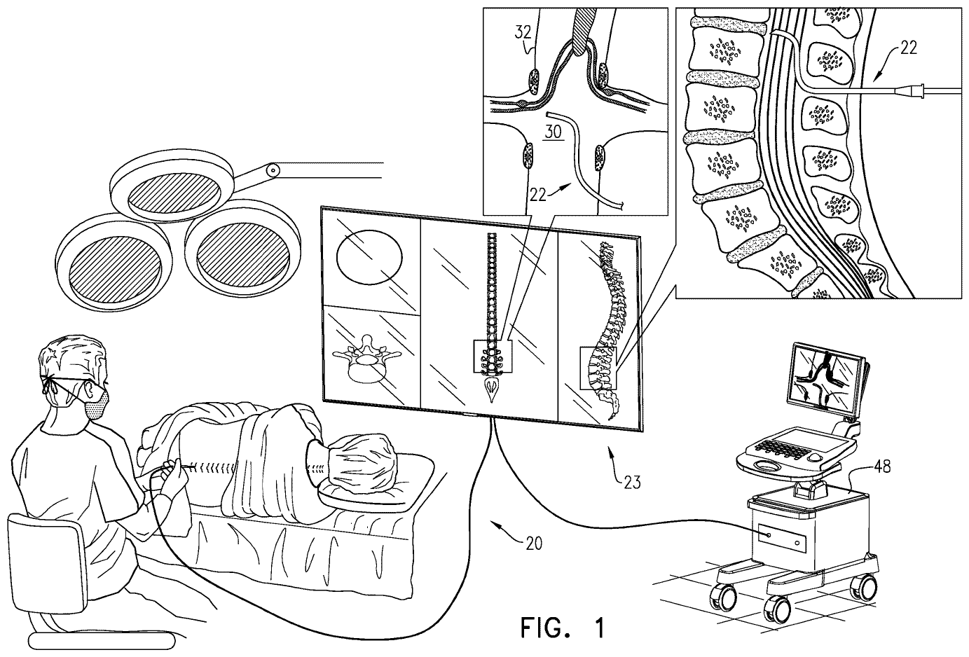

Reference is first made to FIG. 1, which is a schematic illustration of an overview of apparatus 20 comprising diagnostic and operative catheter 22, a distal end of which is configured to be inserted into an intradural space of a subject for diagnosis and/or treatment of spinal and/or cranial pathologies, in accordance with some applications of the present invention. FIG. 1 shows an overview of apparatus 20 including catheter 22 and display unit 23. Catheter 22 is shown being percutaneously inserted into the spine through a lumbar puncture in a lower back of the subject and introduced into intradural space 30 through dural membrane 32 (the lumbar dura). Catheter 22 is then steered within the intradural space to a desired location for the purpose of diagnosis and/or performing a medical procedure for treating a spinal/cranial pathology at the desired location, in accordance with some applications of the present invention.

Apparatus 20 is configured to accurately diagnose real-time pathologies, thus assisting in determining the cause of a spinal/cranial disease. In some cases, imaging modalities, such as MRI, generate indecisive results as to the culprit of the spinal/cranial disease, rendering it difficult to determine which exact pathology (e.g., a pathology of ligaments, bony spurs or discs) are the true disease culprits. In accordance with some applications of the present invention, apparatus 20 is configured to correctly examine, visualize and sample pathological specimens, all under direct, real-time visualization, thereby facilitating accurate diagnosis of the pathology. Additionally, or alternatively, apparatus 20 is configured to treat intradural pathologies by providing effective and targeted treatment of spinal/cranial pathologies through working lumens and ports of catheter 22. For example, apparatus 20 is configured to perform neural ablation of neural elements in the spinal cord and neural foramen. Advantageously, use of apparatus 20 in accordance with applications of the present invention, allows a treating physician to visually examine the pathology while treating the subject.

It is noted that the scope of the present invention includes using the apparatus and methods described herein in anatomical locations other than the intradural space of a subject. Therefore, the intradural catheter and/or portions thereof are sometimes referred to herein (in the specification and the claims) as a catheter or microcatheter.

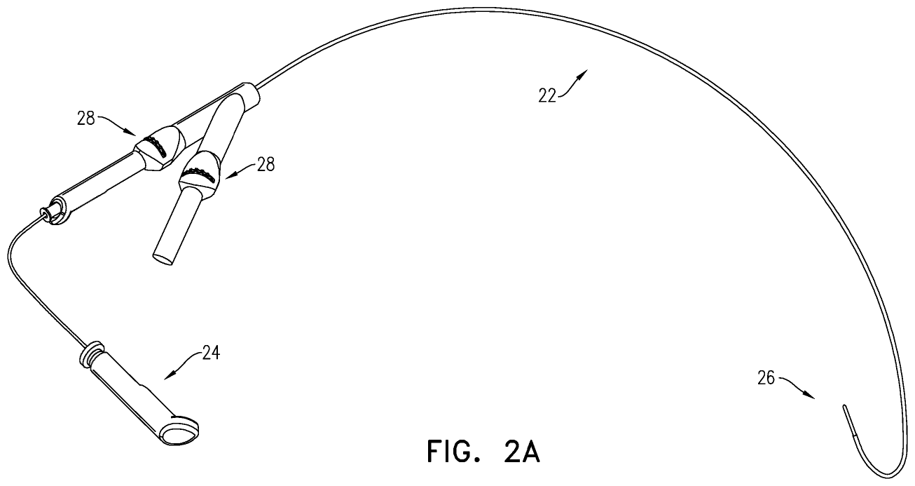

Reference is now made to FIGS. 2A-D, which are schematic illustrations of respective portions of intradural catheter 22 for use in accordance with some applications of the present invention. Catheter 22 typically comprises a flexible steerable catheter that is controlled by a user such as a surgeon or treating physician. As described hereinabove, catheter 22 is typically percutaneously inserted into a lumbar region of the subject, and once inserted, is steered toward a desired target site along the spine. For example, in some cases, catheter 22 is configured to traverse and ascend up to a foramen magnum and a cranial vault of the subject.

Referring first to FIG. 2A, catheter 22 comprises a proximal end 24 and a distal end 26. Proximal end 24 typically comprises a guided sheath including an entry port through which an imaging device and one or more surgical tools can be inserted into the catheter. Distal end 26 typically comprises a deflectable, bendable tip configured to be rotated up to 360 degrees. FIG. 2A additionally show rotators 28 configured to facilitate rotation of distal end 26 up to 360 degrees It is noted that in this context, in the specification and in the claims, “proximal” means closer to the user of the apparatus, and “distal” means farther from the user, and farther into the subject's body from the orifice through which the apparatus is originally placed into the body.

Catheter 22 is typically a multi-lumen catheter comprising at least two lumens, one of which, an optical lumen, is configured for passing an imaging device therethrough and the second lumen, a working lumen terminating with a working port, configured for passing surgical tools, or other treatment modalities, therethrough.

Apparatus 20 is configured to provide real time visualization of the area of the spine (and/or the brain) that is reached by the catheter, as well as providing visualization during navigation of the catheter to the desired location in the spine (and/or the brain). Apparatus 20 typically comprises an imaging device, e.g., a camera, and a light source, which are configured to be passable through the optical lumen of the catheter and to be disposed at distal end 26 of catheter 22. Rotation of deflectable distal end 26 allows rotation of the imaging device and the light source thereby providing visualization of up to 360 angle degrees by the imaging device. The images that are generated by the imaging device may be displayed on display unit 23 shown in FIG. 1.

In addition to providing visualization, apparatus 20 is configured to perform medical procedures on spinal structures for the purpose of diagnosis and/or treatment of spinal pathologies, typically by delivering, through the working lumen of catheter 22, surgical tools to the site of the spinal pathology. The surgical tools may include forceps, an electrosurgery tool, a cutting tool, and/or a suction device, micro-scissors, a pincer, coagulation devices, stents, or any other device to treat any central nervous system pathology.

Both proximal end 24 and distal end 26 contribute to the flexibility of catheter 22. Distal end 26 is bendable and rotatable around itself. Proximal end 24 additionally provides flexibility during steering of the catheter and navigation of the catheter through desired locations (for example, the cranial vault).

For some applications, distal end 26 of catheter 22 comprises magnetic components in the distal tip of the catheter facilitating navigation of the catheter to neural foreman along the spinal canal and up to the cervical spine region. The magnetic components in the distal tip are configured to be imaged by MR and/or CT imaging to detect a location of the distal tip and to generate position data of the magnetic distal end. Alternatively, other navigation/tracking devices and methods are used, e.g., radiopaque markers in distal end 26, fluoroscopy, and/or electrophysiology. In this manner the location of an exact nerve root and/or the vertebral level can be detected during navigation of the catheter.

For some applications, catheter 22 is operatable with a computer processor 48 (shown in FIG. 1) configured to process data, for example imaging data acquired via the imaging device that is inserted via the catheter. For some applications, the computer processor analyzes the imaging data and thereby identifies spinal structures (and/or structures in the brain), in order to assist the physician in guiding the distal end of the catheter toward a given structure. For example, catheter 22 is configured to distinguish between a nerve root and other non-nerve root tissue, thus aiding the physician to perform a medical procedure on the nerve, such as decompression, by using an intradural approach via catheter 22. Additionally, or alternatively, catheter 22 is configured to distinguish between normal tissue and abnormal tissue (e.g., cancerous tissue, an infection or inflammation). For some applications, the computer processor analyzes the imaging data using an artificial intelligence and/or machine-learning algorithm, such as a neural network (e.g., a convolutional neural network), a Linear Regression, Logistic Regression, Decision Tree, Support Vector Machine, Naive Bayes, kNN, K-Means, Random Forest, Dimensionality Reduction Algorithms, and/or Gradient Boosting algorithm.

FIG. 2B is a schematic illustration of distal end 26 of catheter 22 prior to manipulation (e.g., prior to deflection, bending and/or rotation). Distal end 26 is configured to transition between a generally a straight position and a curved position. FIG. 2C is a schematic illustration showing distal end 26 in a curved configuration configured to access spinal structures, such as a neural foramen. FIG. 2D is a schematic illustration showing distal end 26 of catheter 22 having an imaging device 29 and a radiofrequency tip 31 (which can be exchanged with a surgical tool such as micro forceps).

### Neural Foramen Nerve Decompression/Spinal Cord Decompression

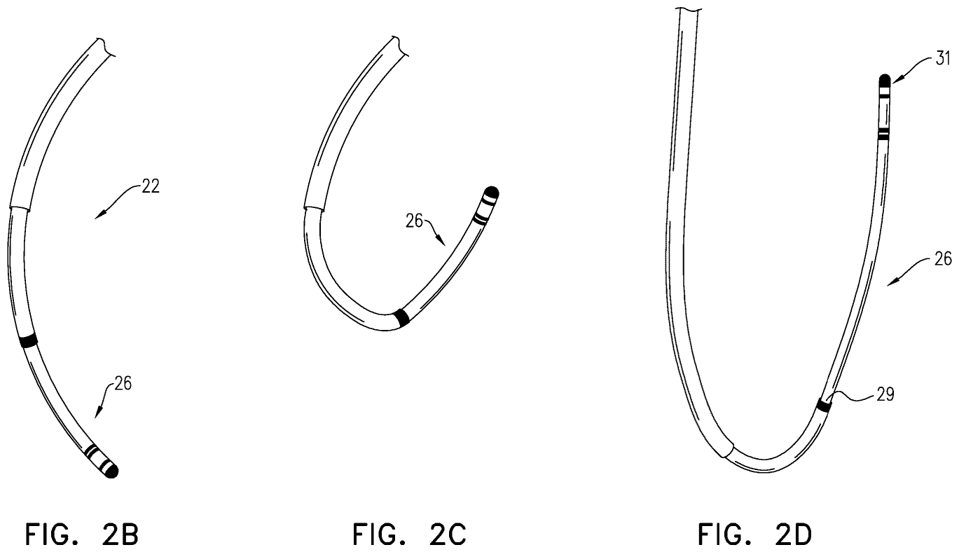

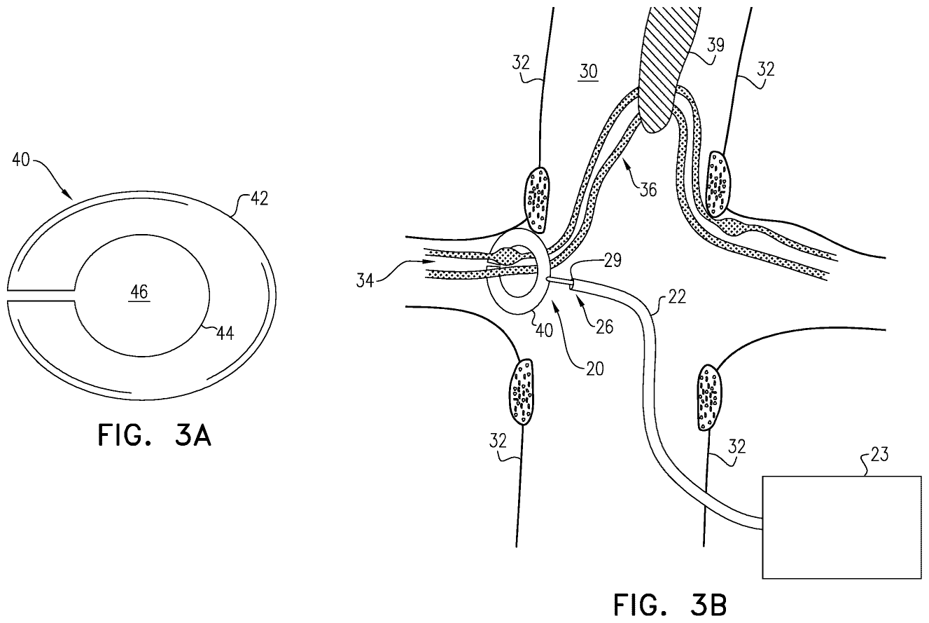

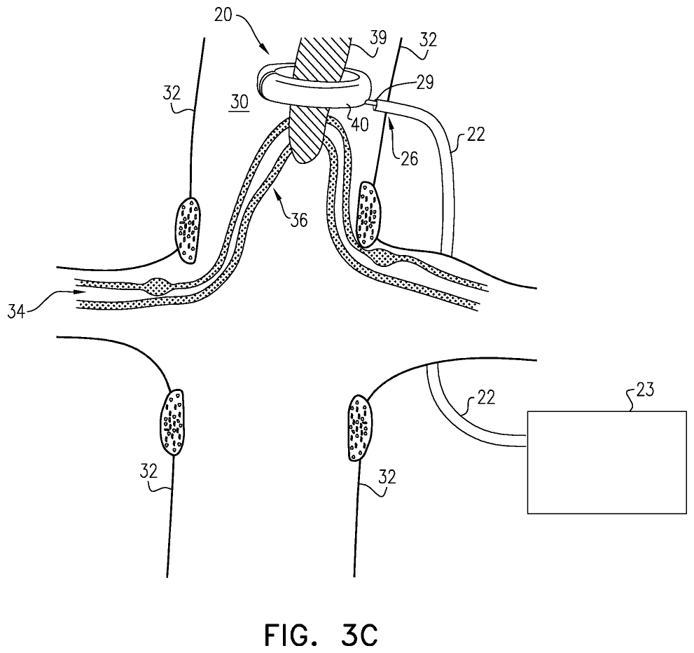

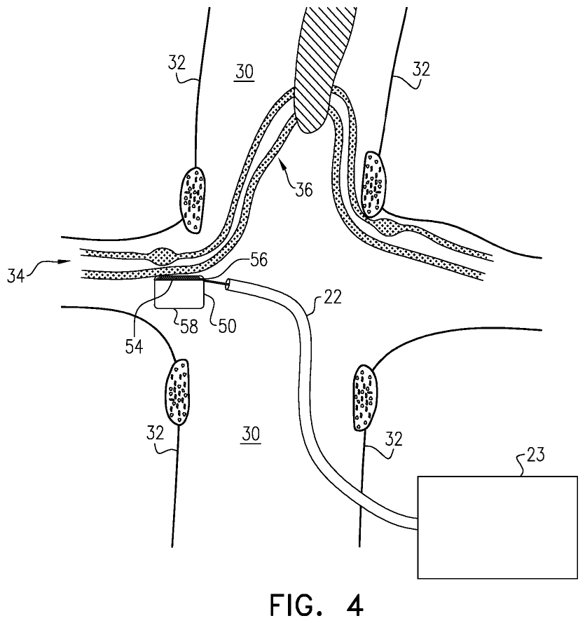

For some applications, apparatus 20 is used to perform neural foramen nerve decompression procedures to treat compressed nerves and provide relief of symptoms associated with compressed nerves. FIGS. 3A-Fig. 4 show configurations of apparatus 20 comprising intradural catheter 22 and an inflatable element, being used to treat compressed nerves, in accordance with applications of the present invention.

Reference is first made to FIGS. 3A and 3B. FIG. 3A is a schematic illustration of an inflatable element 40, which is delivered through intradural catheter 22 for decompression of a neural foramen nerve, as shown in FIG. 3B, in accordance with some applications of the present invention. FIG. 3B is a schematic illustration of a nerve decompression procedure being performed using apparatus 20, in accordance with some applications of the present invention. More specifically, FIG. 3B is a schematic illustration of a neural foramen nerve decompression being performed using apparatus 20 comprising intradural catheter 22 and inflatable element 40 (e.g., a balloon), in accordance with some applications of the present invention.

As described hereinabove, catheter 22 is introduced into intradural space 30 of the subject via a puncturing element (e.g., a needle, e.g., a 2 mm diameter needle), which punctures dural membrane 32, and through which catheter 22 is inserted into the intradural space. Upon insertion, catheter 22 is navigated to the neural foramen, and while under vision by imaging device 29, inflatable element 40 is advanced out of distal end 26 of catheter 22 to at least partially surround nerve 34, in the neural foramen, and decompress the nerve via inflation of inflatable element 40 around the nerve, while protecting the nerve itself (by not applying any pressure in the direction of the nerve). (In FIG. 3B spinal nerves 34 and nerve root 36 are indicated).

Inflatable element 40 is delivered through the working lumen of catheter 22 in a collapsed state. When the inflatable element is disposed at a distal end of intradural catheter 22 and the catheter is at the desired location in the vicinity of nerve 34, the inflatable element is advanced out of the distal end of the catheter and is inflated, through the intradural catheter, from the collapsed state to an inflated state. In the inflated state, shown in FIGS. 3A-B, inflatable element 40 is shaped to define an outer wall 42, an inner wall 44 and a cavity 46 at least partially surrounded by inner wall 44. As shown in FIGS. 3A-B, for some applications, inflatable element 40 is inflated to define a toroidal (ring-shaped) inflatable element. For some applications, inner wall 44 of inflatable element 40 is shaped to define an inner diameter of the inflatable element that is 0.2-0.5 cm, e.g., 0.3-0.4 cm, and outer wall 42 is shaped to define an outer diameter of the inflatable element that is 0.5-1 cm, e.g., 0.6-0.8 cm.

As shown in FIG. 3B, inflatable element 40 is inflated around nerve 34 such that the nerve is disposed in cavity 46 surrounded by inner wall 44 of inflatable element 40. Outer wall 42 of inflatable element 40 is inflated outwardly to apply pressure in a direction that is away from the nerve, thereby decompressing the nerve while protecting the nerve. Typically, only outer wall 42 is inflated outwardly, while inner wall 44 is not inflated outwardly, thus preventing pressure being applied by inner wall 44 to nerve 34. Typically, inflatable element 40 is inflated around the nerve such that the inner wall 44 is placed at a distance of about 3 mm from the nerve.

Reference is now made to FIG. 3C, which is a schematic illustration of a medical procedure being performed with respect to spinal cord 39 using apparatus 20 comprising intradural catheter 22 and inflatable element 40, in accordance with some applications of the present invention. As described hereinabove with respect to FIG. 3B, inflatable element 40 is delivered through intradural catheter 22 and inflated around a target site. In the procedure illustrated in FIG. 3C, inflatable element 40 is inflated around spinal cord 39. For some such applications, inflatable element 40 is configured to expand the diameter of the spinal canal and reduce pressure from spinal cord 39. Typically, inflatable element 40 is configured to expand the diameter of the spinal canal in cervical, thoracic, and/or lumbar regions of the spine.

It is noted that deployment and inflation of inflatable element 40, as described in FIGS. 3B-C, can be performed in an outpatient setting in cases in which there are suspected cases of nerve or spinal cord compression (e.g., in cases of suspected spinal cord trauma injury involving spinal cord compression).

Reference is now made to FIG. 4, which is a schematic illustration of a nerve decompression procedure being performed using apparatus 20, in accordance with another application of the present invention. More specifically, FIG. 4 is a schematic illustration of a neural foramen nerve decompression being performed using intradural catheter 22 and an inflatable element 50 (e.g., a balloon), in accordance with some applications of the present invention. For some applications, as shown in FIG. 4, inflatable element 50 comprises one or more electrodes 54 configured to sense and monitor neural activity of nerve 34. Inflatable element 50 is delivered through catheter 22 and advanced out of the distal end of the catheter in proximity to compressed nerve 34 in the vicinity of the neural foramen. In the inflated state, inflatable element 50 is typically shaped to define a near side 56 and a far side 58.

Inflatable element 50 is inflated through catheter 22, in the neural foramen in proximity to nerve 34, such that near side 56 of inflatable element 50 faces portions of nerve 34, and far side 58 of inflatable element 50 faces away from nerve 34. Far side 58 is inflated outwardly to apply pressure in a direction that is away from nerve 34, thereby decompressing the nerve. Near side 56 generally does not apply pressure to the nerve. Electrode 54 that is coupled to inflatable element 50 senses and monitors nerve activity of nerve 34. In response to detecting the nerve activity, and thereby determining the location of the nerve, near side 56 of inflatable element 50 is positioned such as to face the nerve. Typically, the electrode is disposed on the near side of the inflatable element, such that upon the electrode detecting the nerve activity, the near side of the inflatable element is facing the nerve.

### Intra-Ganglion Balloon Compression

For some applications, apparatus 20 is used to perform ganglion compression procedures to selectively affect portions of a nerve, e.g., a ganglion, that convey pain signals to the brain, to thereby to disrupt pain signals from getting through to the brain. For some applications, the ganglion is destroyed.

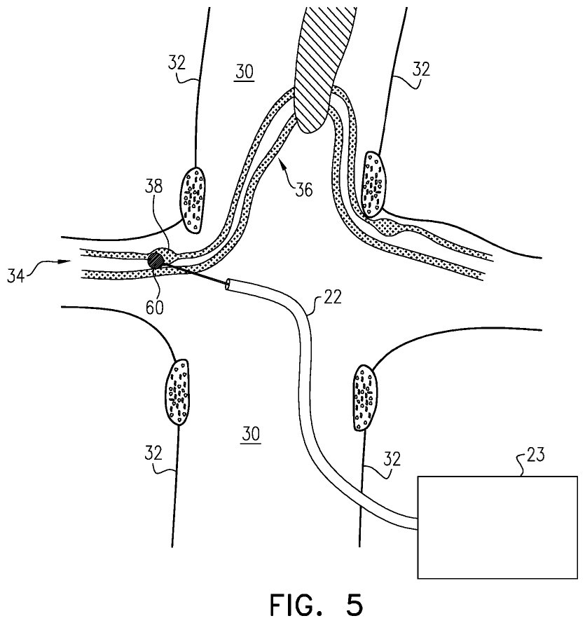

Reference is now made to FIG. 5, which is a schematic illustration of a ganglion compression procedure being performed using apparatus 20, in accordance with some applications of the present invention. As shown in FIG. 5, for some such applications, apparatus 20 comprises intradural catheter 22 and an inflatable element 60. Inflatable element 60 is delivered through catheter 22 and advanced out of the distal end of the catheter in proximity to ganglion 38 of nerve 34 in the vicinity of the neural foramen. In the inflated state inflatable element 60 compresses ganglion 38 to help with pain relief, in accordance with applications of the present invention.

In a similar procedure, apparatus 20 is used to perform an intracranial nerve rhizotomy, in accordance with techniques described herein.

### Tumor Treatment

For some applications, apparatus 20 is used to perform medical procedures involving a tumor, e.g., an intradural tumor. For example, apparatus 20 is used to obtain a biopsy sample of the tumor, and/or removal of the tumor. In accordance with some applications of the present invention, apparatus 20 is configured for obtaining brain and/or spine biopsies in a minimally invasive manner via an intradural approach, as described herein.

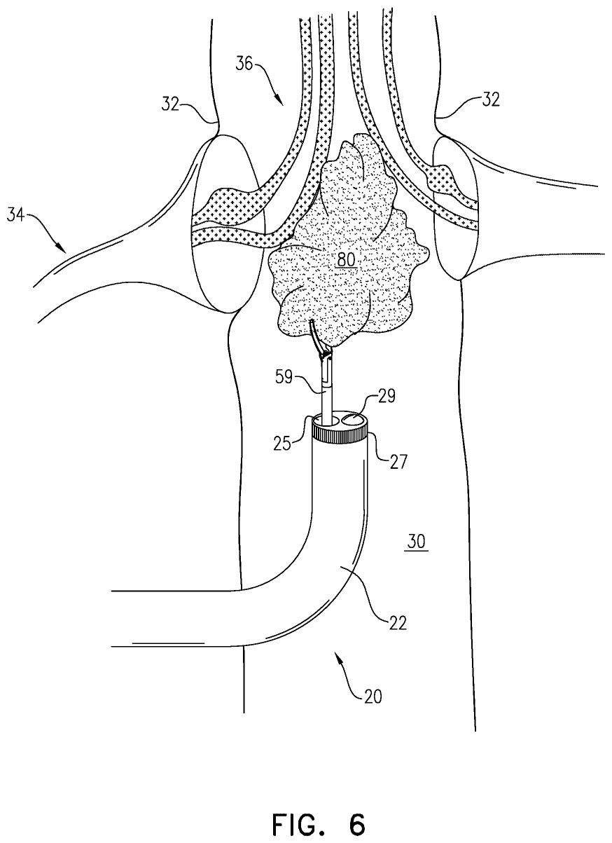

Reference is now made to FIG. 6, which is a schematic illustration of apparatus 20 accessing an intradural tumor 80 for performing a medical procedure, in accordance with some applications of the present invention. As shown, for some such applications, apparatus 20 comprises intradural catheter 22 and a surgical tool 59 (e.g., micro tumor forceps and/or a coagulation device). Surgical tool 59 is delivered through working lumen 25 of catheter 22 and advanced out of the distal end of the catheter in proximity to tumor 80 in intradural space 30. Typically, navigation of catheter 22 to tumor 80 and performing the medical procedure on tumor 80, are carried out under direct visualization by imaging device 29. As described hereinabove, navigation of apparatus 20 is typically facilitated by a magnetic component 27 (and/or a different navigation aiding component) disposed at the distal end of catheter 22.

As described hereinabove, for some applications, catheter 22 is operatable with computer processor 48, which is configured to process data, for example imaging data acquired via imaging device 29. For some applications, using artificial intelligence and/or machine-learning algorithms the computer processor analyzes the imaging data received from the imaging device to identify a tumor and/or identify characteristics of the tumor. For example, catheter 22 is configured to distinguish between different types of tumors, thus aiding the physician to perform the appropriate medical procedure on the tumor, such as dissection and/or application of a treating agent. For some applications, the computer processor is configured to create a bank of tumor/abnormal tissue data by using recordings from the images acquired by imaging device 29 (or other visual means such as spectroscopy). For some such applications, apparatus 20 is trained to diagnose tumors by viewing them under direct visualization and using an artificial intelligence and/or machine-learning algorithm, such as a neural network (e.g., a convolutional neural network), a Linear Regression, Logistic Regression, Decision Tree, Support Vector Machine, Naive Bayes, kNN, K-Means, Random Forest, Dimensionality Reduction Algorithms, and/or Gradient Boosting algorithm.

### Microvascular Decompression

For some applications, apparatus 20 is used to perform microvascular decompression procedures to move an offending blood vessel away from an affected nerve. Current microvascular decompression surgical techniques for trigeminal neuralgia or other cranial neuropathies, typically involve an open craniotomy, anesthesia and a neurotreating physician. Typically, a medical divider, such as a polytetrafluoroethylene pad, is inserted between the offending blood vessel and the inflicted nerve via craniotomy.

In contrast, and in accordance with some applications of the present invention, apparatus 20 and methods described herein are used for performing microvascular decompression in a minimally invasive approach through a lumbar puncture. For some such applications, a catheter as described herein is introduced to the body of the subject via a needle which punctures dura, and through which the catheter is inserted into the spinal canal. As described hereinabove, the catheter has a working port and an imaging device at the distal end thereof. The distal end of the catheter is fully controlled by the user and is configured to be revolved around itself 360 degrees (approximately 4 cm proximally to the tip of catheter at the distal end).

Upon insertion into the spinal canal, the catheter is navigated to a brainstem of the subject, using navigation techniques described hereinabove. The catheter is brought into proximity to the relevant cranial nerves, and the area requiring microvascular decompression is identified. Medical dividers such as polytetrafluoroethylene sheets, are advanced out of the catheter (the medical dividers may be held by graspers that are also delivered through the working lumen of the catheter). Under direct visualization, the medical divider is placed between the relevant cranial nerve in conflict with a vascular structure, thereby buffering the nerve from the pulsations of the blood vessels.

Additionally, or alternatively, the catheter is configured to perform microvascular decompression, and to deliver an inflatable/expandable stent via the working port of the catheter. The inflatable stent is inserted between the nerve and the offending blood vessel.

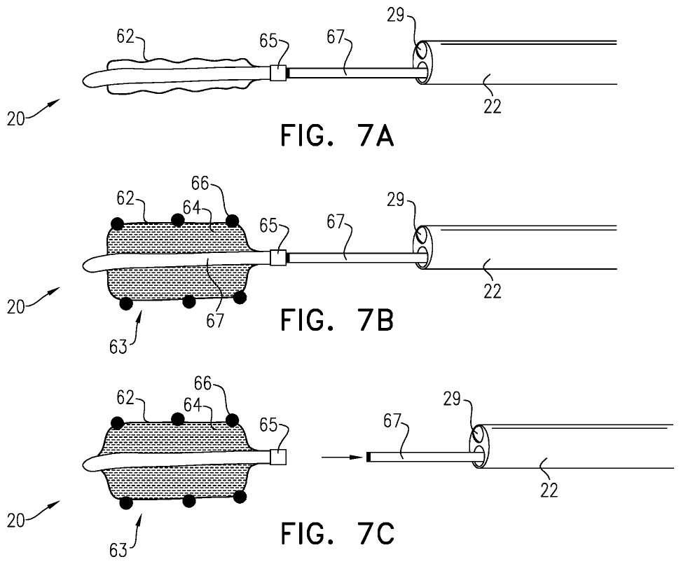

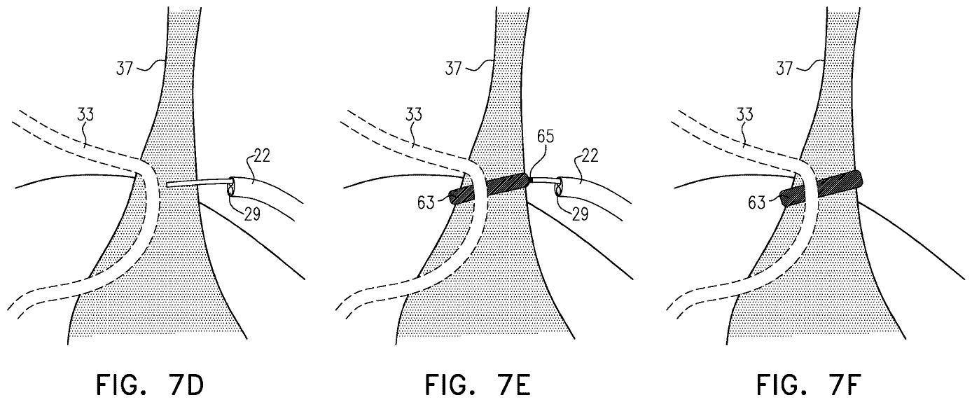

Reference is now made to FIGS. 7A-7F, which are schematic illustrations of an example of microvascular decompression surgical apparatus and technique, in accordance with some applications of the present invention. More specifically, FIGS. 7A, 7B, and 7C are schematic illustrations of apparatus 20 comprising intradural catheter 22 and a medical divider 63, in accordance with some applications of the present invention. FIGS. 7D, 7E and 7F are schematic illustrations of a microvascular decompression medical procedure being performed using apparatus 20 comprising intradural catheter 22 and medical divider 63, in accordance with some applications of the present invention.

Apparatus 20, shown in FIGS. 7A-7F, is used for performing microvascular decompression in a minimally invasive approach as described herein. Typically, catheter 22 is introduced into the body of the subject via a needle which punctures dura, and through which the catheter is inserted into the spinal canal. Upon insertion into the spinal canal, catheter 22 is navigated to a brainstem of the subject, under direct visualization, using navigation techniques described hereinabove. Catheter 22 is brought into proximity to the relevant cranial nerves, and the area requiring microvascular decompression is identified. Under direct visualization, medical divider 63 is inserted between a relevant cranial nerve 37 and a vascular structure 33 (e.g., a blood vessel such as an artery or a vein), as will be described in further detail below. Medical divider 63 is a mechanical separator, which when placed between cranial nerve 37 and a vascular structure 33 buffers the nerve from the pulsations of the blood vessels.

For some applications, medical divider 63 comprises an expandable element 62 (such as an inflatable balloon) and a generally solid dividing substance such as Polytetrafluoroethylene. Medical divider 63 is configured to be delivered and deployed by catheter 22 between cranial nerve 37 and a vascular structure 33.

FIG. 7A, shows catheter 22 having imaging device 29 and a working port through which delivery lumen 67 is advanced toward relevant cranial nerves in the area requiring microvascular decompression. Expandable element 62 is disposed at a distal end of delivery lumen 67 of catheter 22. In FIG. 7A, expandable element 62 is shown in a collapsed state thereof. Typically, the polytetrafluoroethylene is shaped and sized to be delivered through delivery lumen 67 towards expandable element 62 (the polytetrafluoroethylene is not shown in FIG. 7A, for clarity). Typically, a fluid, e.g., saline, is advanced through catheter 22 and delivery lumen 67, thereby advancing the polytetrafluoroethylene distally in lumen 67 and into expandable element 62.

As shown in FIG. 7B, polytetrafluoroethylene (indicated by reference numeral 64) is distributed into the interior of expandable element 62, while the saline (not shown) that is advanced through delivery lumen 67 and into expandable element 62, exits expandable element 62 through pores in expandable element 62 (leaving expandable element 62 in the expanded state). The pores of expandable element 62 are indicated in FIGS. 7C and 7B by reference numeral 66. Expandable element 62 is shown as having six pores by way of illustration and not limitation. It is noted that expandable element 62 may be shaped to define any suitable number of pores, which are generally shaped and sized to allow passage of a fluid such as saline therethrough, on the one hand, and to maintain the polytetrafluoroethylene within expandable element 62, on the other hand. As shown in FIG. 7B, when the polytetrafluoroethylene is disposed in expandable element 62, medical divider assumes an operatable, generally stable, configuration for separating between the nerve and the blood vessel.

Medical divider 63 is typically disconnected from catheter 22 such that divider 63 remains as a separator between a nerve and a blood vessel, while catheter 22 is retracted from the body of the subject. Disconnection of medical divider 63 from catheter 22 is typically done by application of high frequency electrical current to disconnection zone 65, shown in FIG. 7C.

Reference is again made to FIGS. 7D, 7E and 7F, which, as described above, are schematic illustrations of a microvascular decompression medical procedure being performed using apparatus 20 comprising intradural catheter 22 and medical divider 63, in accordance with some applications of the present invention.

As shown in FIG. 7D, catheter 22 is brought into proximity to cranial nerve 37 and vascular structure 33 (i.e., a blood vessel). Medical divider 63 is then disposed between nerve 37 and vascular structure 33 thereby mechanically separating nerve 37 from the pulsations of vascular structure 33 (FIG. 7E). Subsequently to placement of divider 63, divider 63 is disconnected from catheter 22 at disconnection zone 65 (typically by application of electrical current), and catheter 22 is retracted and removed from the body. Medical divider 63 remains in place to separate between nerve 37 and vascular structure 33, as shown in FIG. 7F. (Medical divider 63 functions similar to a stent that is expanded between the nerve and the vascular structure).

### Additional Diagnostic and Therapeutic Applications

In accordance with some applications of the present invention, apparatus 20 and techniques described herein with reference to FIGS. 1-7F, are used in additional diagnostic and therapeutic applications.

For example, apparatus 20 and techniques described herein are configured for use to perform any one of the following diagnostic and/or therapeutic procedures: performing a biopsy of the brain and spinal cord, performing a biopsy from the spinal canal/brain tissue via a lumbar puncture, visualizing neural structures of the brain and the spine in real time, placement of EMG electrodes and/or other monitoring devices in the brain and spinal canal, administration of drugs via a lumbar puncture approach, placement of electrodes in the brain and/or spinal canal, treatment of intra-spinal lesions, treatment of syringomyelia, fenestration of spinal arachnoiditis/arachnoid cysts both in the spine and intracranially, treatment of intracranial lesions, brainstem cerebellopontine (CPA) lesions such as tumors/epidermoids and other CPA lesions, diagnosis and repair of cerebrospinal fluid leaks, diagnosis of subarachnoid hemorrhage occurring intracranially, access and treat intracranial aneurysms by placement of a clip or wrapping of a sheath around the aneurysm under vision (i.e., basilar tip aneurysms), perform radiofrequency/rhizotomy/injections to the neural foramen, treatment of Chiari malformations, diagnosis and potential repair of spinal dural defects, diagnosis of spinal cord herniation, diagnosis of malfunction/malposition of a lumboperitoneal shunt and/or a ventriculoperitoneal shunt, third ventriculostomy procedures, choroid plexus cauterization, aqueductal plasty procedures, treatment of aqueductal stenosis, treatment of hydrocephalus, and postoperative evaluation of subjects who have undergone brain and spinal surgery.

It is additional noted that use of apparatus 20 and techniques practiced in accordance with applications of the present invention may be used to replace MRI for diagnostic purposes. For example, for diagnosis of intradural or degenerative spine conditions (i.e., spinal stenosis). Additionally, or alternatively, apparatus 20 and techniques practiced in accordance with applications of the present invention may be used in cases of in inconclusive diagnostic spine MRI, by providing accurate three-dimensional real-time diagnosis of spinal pathology.

It is further noted that that apparatus 20 and techniques practiced in accordance with applications of the present invention, are particularly advantageous for use in subject that cannot undergo myelogram, or MRI or any procedures using of contrast agents, subjects in whom MRI is contraindicated, subjects suffering from claustrophobia, and/or subjects suffering from renal failure and cannot tolerate administration of gadolinium.

Reference is again made to apparatus 20 and techniques described herein with reference to FIGS. 1-7F. It is noted that apparatus 20 is configured for use as a stand-alone device. Additionally, or alternatively, apparatus 20 and techniques described herein are configured for use in combination with electrophysiology neuromonitoring and/or with the use of a fluorescence device intraoperatively to detect exact lumbar levels and navigate accordingly in real time.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 48. For the purposes of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 48) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that algorithms described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 48) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 48 is typically a hardware device programmed with computer program instructions to produce a special purpose computer. For example, when programmed to perform the algorithms described herein, computer processor 48 typically acts as a special purpose spinal-treatment computer processor. Typically, the operations described herein that are performed by computer processor 48 transform the physical state of a memory, which is a real physical article, to have a different magnetic polarity, electrical charge, or the like depending on the technology of the memory that is used.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus, comprising:
an intradural catheter configured to be passed into a spinal canal of the subject and to be advanced toward a compressed nerve in the spinal canal; and
an inflatable element disposed at a distal end of the intradural catheter and configured to be inflated from a collapsed state to an inflated state, such that when in the inflated state the inflatable element is shaped to define an outer wall, an inner wall and a cavity at least partially surrounded by the inner wall;
wherein the inflatable element is configured to be inflated, through the intradural catheter, around the nerve such that (a) the nerve is disposed in the cavity surrounded by the inner wall of the inflatable element, and (b) the outer wall of the inflatable element is inflated outwardly to apply pressure in a direction that is away from the nerve.

2. The apparatus according to claim 1, wherein the inflatable element comprises a toroidal inflatable element.

3. The apparatus according to claim 1, wherein the inflatable element comprises an inflatable balloon.

4. The apparatus according to claim 1, wherein the inner wall of the inflatable element is shaped to define an inner diameter of the inflatable element that is 0.3-0.4 cm.

5. The apparatus according to claim 1, wherein the outer wall is shaped to define an outer diameter of the inflatable element that is 0.6-0.8 cm.

6. The apparatus according to claim 1, wherein the apparatus further comprises an imaging device disposed at the distal end of the intradural catheter and configured to provide visualization of an area of the spine that is treated by the apparatus.

7. The apparatus according to claim 1, wherein the intradural catheter comprises a flexible catheter and wherein the distal end of the intradural catheter comprises a bendable tip configured to be rotated up to 360 degrees.

8. The apparatus according to claim 1, wherein the intradural catheter is configured to allow passage therethrough of a surgical tool selected from the group consisting of: forceps, an electrosurgery tool, a cutting tool, and a suction device.

9. The apparatus according to claim 1, wherein the apparatus further comprises a puncturing element configured to puncture spinal dura of the subject to allow the intradural catheter to be passed into the spinal canal, the catheter being configured to be passable through the puncturing element.

10. The apparatus according to claim 1, wherein the intradural catheter is configured to be passed into the spinal canal of the subject and to be advanced toward the compressed nerve in a vicinity of a neural foramen in the spinal canal.

11. The apparatus according to claim 1, wherein the intradural catheter is configured to be passed into the spinal canal of the subject and to be advanced toward the compressed nerve in a vicinity of a spinal cord.

12. The apparatus according to claim 1, wherein the distal end of the intradural catheter comprises a magnetic distal tip configured to be imaged by MR and/or CT imaging to detect a location of the distal tip.

13. A method for decompressing of a nerve of a subject, the method comprising:
inserting into a spinal canal of a subject, an intradural catheter that includes an inflatable element disposed at a distal portion of the intradural catheter;
advancing the intradural catheter toward a compressed nerve in a vicinity of a neural foramen in the spinal canal;
inflating, through the intradural catheter, the inflatable element around the nerve such that: (a) the nerve is disposed in a cavity that is at least partially surrounded by an inner wall of the inflatable element, and (b) an outer wall of the inflatable element is inflated outwardly to apply pressure in a direction that is away from the nerve, thereby decompressing the nerve.

14. The method according to claim 13, wherein inflating the inflatable element comprises inflating the inflatable element to define a toroidal shape.

15. The method according to claim 13, wherein inflating the inflatable element around the nerve comprises inflating the inflatable element such that pressure applied by the outer wall is greater than pressure applied by the inner wall.

16. The method according to claim 13, wherein inflating the inflatable element around the nerve comprises inflating the inflatable element such that the inner wall does not apply pressure to the nerve.

17. The method according to claim 13, wherein inflating the inflatable element around the nerve comprises inflating the inflatable element such that the inner wall is placed at a distance of 3 mm from the nerve.

18-27. (canceled)

28. An apparatus, comprising:
an intradural catheter configured to be passed into a spinal canal of the subject and to be advanced toward a nerve in a vicinity of a neural foramen in the spinal canal;
an inflatable element disposed at a distal end of the intradural catheter and configured to be inflated from a collapsed sate to an inflated state in the neural foramen, such that in the inflated state the inflatable element is shaped to define a near side and a far side; and
at least one electrode coupled to the inflatable element and configured to sense nerve activity of the nerve;
wherein the inflatable element is configured to be inflated, in proximity to the nerve such that (a) the near side faces the nerve and (b) the far side faces away from the nerve, the far side being configured to be inflated outwardly to apply pressure in a direction that is away from the nerve.

29. The apparatus according to claim 28, wherein the electrode is coupled to the near side of the inflatable element.

30. The apparatus according to claim 28, wherein when in the inflated state the near side of the inflatable element does not apply pressure to the nerve.

31. (canceled)
